(19) United States
(12) Patent Application Publication
(10) Pub. No.: US 2021/0406731 A1
(43) Pub. Date: Dec. 30, 2021

(54) NETWORK-IMPLEMENTED INTEGRATED MODELING SYSTEM FOR GENETIC RISK ESTIMATION

(71) Applicant: InheRET, Inc., Ann Arbor, MI (US)

(72) Inventors: David Keren, Ann Arbor, MI (US); Sofia Merajver, Ann Arbor, MI (US); Lynn McCain, Milan, MI (US); Amanda Cook, Gregory, MI (US); Lee Schroeder, Ann Arbor, MI (US); Kara Milliron, Ann Arbor, MI (US)

(73) Assignee: InheRET, INC., Ann Arbor, MI (US)

(21) Appl. No.: 17/138,319

(22) Filed: Dec. 30, 2020

Related U.S. Application Data
(60) Provisional application No. 63/046,498, filed on Jun. 30, 2020.

(51) Int. Cl. G06N 5/04; G06N 5/02; G16H 50/30; G16H 10/60; G16H 70/20

(52) U.S. Cl. CPC G06N 5/04; G06N 5/02; G16H 70/20; G16H 10/60; G16H 50/30

(57) ABSTRACT

A network-implemented integrated modeling system for genetic risk estimation uses guideline factor information and other input information to determine models usable for estimating genetic risk of disease in patients. Models are determined using established network connections with external sources. Health history information is collected from a patient, which is then processed against a relevant model to estimate the genetic risk of a disease corresponding to that model in that patient. Based on results of the estimation, a pedigree chart representing personal and family risk for the patient is generated and output to the patient and/or health care representatives of the patient. The results may be disseminated to one or more distributed computing environments from a central server which performs the genetic risk estimation. In some cases, follow-up validations performed using further collected data are used to update the constraints used to determine the models.

### CROSS-REFERENCE TO RELATED APPLICATION(S)

This application claims priority to and the benefit of U.S. Provisional Application No. 63/046,498, filed Jun. 30, 2020, the entire disclosure of which is incorporated by reference herein for all purposes.

### TECHNICAL FIELD

This disclosure relates to a network-implemented integrated modeling system for genetic risk estimation, in particular, to an integrated system for estimating genetic risk using network-implemented software that models genetic risk using collected factors.

### BACKGROUND

Research into human genetics has shown certain disease susceptibilities in patients based on the presence of certain health and family factors of those patients. For example, it is believed that millions of people are at increased risk of cancer due to family history factors. This is understood to be as a result of the genetic information passed to those persons and their family members. Existing guidelines may be useful, although not necessarily always dispositive, to assess an individual's genetic risk for a specific disease.

### SUMMARY

In one implementation, a network-implemented integrated modeling system for genetic risk estimation comprises a server device implementing a web application accessible over a network by a client device and a database storing information including a first set of guideline factor information obtained from one or more external sources and a second set of guideline factor information developed for the web application. The web application is configured to: transmit, over the network, a request for personal and family health information associated with a patient to the client device; receive, over the network, the personal and family health information from the client device; estimate, at the server device, a genetic risk of the patient for one or more specified diseases using at least some of the personal and family health information and using models determined, at the web application, for estimating genetic risk of the one or more specified diseases using one or both of the first guideline factor information or the second guideline factor information; generate, at the server device, a pedigree chart and reporting data based on results of the genetic risk estimation, the pedigree chart visually representing relationships between the patient and family members of the patient, the reporting data indicating actions for the patient to take based on the results of the genetic risk estimation; and distribute, over the network, the pedigree chart and the reporting data to the client device.

In another implementation, network-implemented integrated modeling system for genetic risk estimation comprises a server device implementing a web application accessible over a network by a client device and a database storing information including guideline factor information. The web application is configured to: determine a model for a specified disease using manifestations, fields, and filters derived from the guideline factor information; collect, from the client device, personal and family health information associated with a patient defined based on the manifestations, fields, and filters of the model; estimate a genetic risk of the patient for the specified disease by applying the personal and family health information against the model; generate a pedigree chart and reporting data based on results of the genetic risk estimation; and distribute the pedigree chart and the reporting data to the client device.

In yet another implementation, network-implemented integrated modeling system for genetic risk estimation comprises a server device implementing a web application and a database storing information including first guideline factor information obtained from one or more external sources and second guideline factor information developed for the web application, in which the web application is accessible over a network by each of a first client device associated with a patient and a second client device associated with a health care provider who provides health care service to the patient. The web application is configured to: estimate a genetic risk of the patient for a specified disease by applying personal and family health information associated with the patient against a model determined at the web application using one or both of the first guideline factor information or the second guideline factor information; generate, at the server device, results of the genetic risk estimation indicating whether further genetic counseling or testing for the specified disease is recommended; and distribute, over the network, the results of the genetic risk estimation to the first client device and to the second client device.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram of an example of a system for network-implemented integrated modeling for genetic risk estimation.

FIG. 2 is a block diagram showing examples of application and database functionality of software of a system for network-implemented integrated modeling for genetic risk estimation.

FIG. 3 is a block diagram of an example workflow of a network-implemented integrated modeling system for genetic risk estimation.

FIG. 4 is a block diagram of an example of inputs and criteria used for modeling genetic risk.

FIG. 5 is an illustration of a pedigree chart generated for a patient.

FIG. 6 is a flowchart of an example of a technique for genetic risk estimation.

FIG. 7 is a block diagram of an example internal configuration of a computing device of a system for network-implemented integrated modeling for genetic risk estimation.

### DETAILED DESCRIPTION

Health care providers who suspect their patients of being at increased risk for certain diseases may recommend preventative measures or treatments to mitigate the potential onset of those diseases. For example, for a patient who has a family history of colorectal cancer, meaning that one or more of his or her family members has been diagnosed with colorectal cancer, and who is classified as obese due to his or her weight, a recommendation may be made to alter his or her diet to include and/or exclude certain food types to decrease the statistical likelihood of that patient later developing colorectal cancer. For some individuals with an inherited susceptibility to disease, risk reducing surgeries can in some cases reduce their risk of being diagnosed with a given disease by nearly 100 percent. However, in general, fewer than 10 percent of patients know they are at risk for inherited susceptibility to disease.

Several barriers exist which inhibit a patient's ability to become aware of their genetic risk for disease. Examples of such barriers include, but are not necessarily limited to, the constrained amount of time a patient spends with a clinician, anxiety and/or misunderstandings arising in filling out family history forms, clinical errors in patient care such as by an incorrect lab test being ordered, and issues with insurance pre-authorization which may delay or entirely prevent a person from seeking appropriate medical care. Ultimately, unless a person specifically seeks out genetic counseling to estimate his or her genetic risk for disease, these and/or other barriers will cause them to not know that they carry such genetic risks.

Given the greater health care industry's adoption of electronic medical records, one conventional solution operating in a computing environment may be to use information already present within an electronic patient record to estimate that patient's genetic risk for disease. For example, a system may be configured to parse electronic patient records for certain data entries, such as keywords, commonly associated with certain diseases. Electronic patient records having those data entries may be flagged, such as to alert the clinician who keeps those records as to the potential disease risk that the respective patients have.

This approach has numerous drawbacks, from both technical limitations of the systems and inefficiencies in the data processing. For example, health care providers may not have access to tools for which genetic risk estimation for hereditary diseases has been tested and validated. In another example, an estimation of genetic risk based on a patient's clinical record alone may be materially incomplete. That is, the personal history of the patient which is actually documented in the record may represent only a recent period of the patient's life. Even to the extent the record is kept up to date by a clinician who has cared for the patient for his or her entire life, the record may often omit critical family history factors which research has proven to be highly relevant to genetic risk estimation. This may be due, for example, to the patient never having discussed family health histories with the clinician and/or due to forms filled out by the patient lacking fields for collecting extensive family health histories which are required for accurate assessments of the risk of hereditary diseases or syndromes.

A major failure in conventional genetic risk estimation methods thus arises from the limited technical abilities of such methods, such as due to limitations in the abilities of such technical approaches to extract and/or develop critical guideline information, to model important factors indicated by the guideline information to assess patient personal and family health information, and to deploy a distributed platform accessible by patients and clinical patient representatives to collect extensive history information by leveraging a network-based approach to deliver and receive information.

Implementations of this disclosure address problems such as these by using a network-implemented integrated modeling system for genetic risk estimation. In particular, the implementations of this disclosure teach a network-implemented integrated modeling system for genetic risk estimation which uses guideline factor information and other input information to determine models usable for estimating genetic risk of disease in patients. Models are determined using guideline factor information obtained from external sources and/or developed for the system. Health history information is collected from a patient, which is then processed against a relevant model to estimate the genetic risk of a disease corresponding to that model in that patient. Based on results of the estimation, a pedigree chart representing family relationships revealed by the health history information is generated. An output report is also generated and distributed to the patient and/or health care representatives of the patient. The results may be disseminated to one or more distributed computing units from a central server which assesses the genetic risk estimation and determines further actions based on the assessment. In some cases, follow-up validations performed using further collected data may be used to update the constraints used to determine the models.

As used herein, the term “patient” refers to a person who uses a network-implemented integrated modeling system for genetic risk estimation as disclosed herein for the purpose of obtaining an informed estimate of his or her genetic risk for disease, regardless of whether or not that person has previously sought, or intends to seek, medical evaluation, genetic counseling, or like assistance related or unrelated to any diseases for which his or her genetic risk is estimated. Thus, while a patient as described herein may in some cases be a person who receives a referral or recommendation to use a network-implemented integrated modeling system for genetic risk estimation as disclosed herein by a health care provider, a patient as described herein may in other cases instead be a person who seeks genetic risk estimation independent of a referral or recommendation to do so by a health care provider.

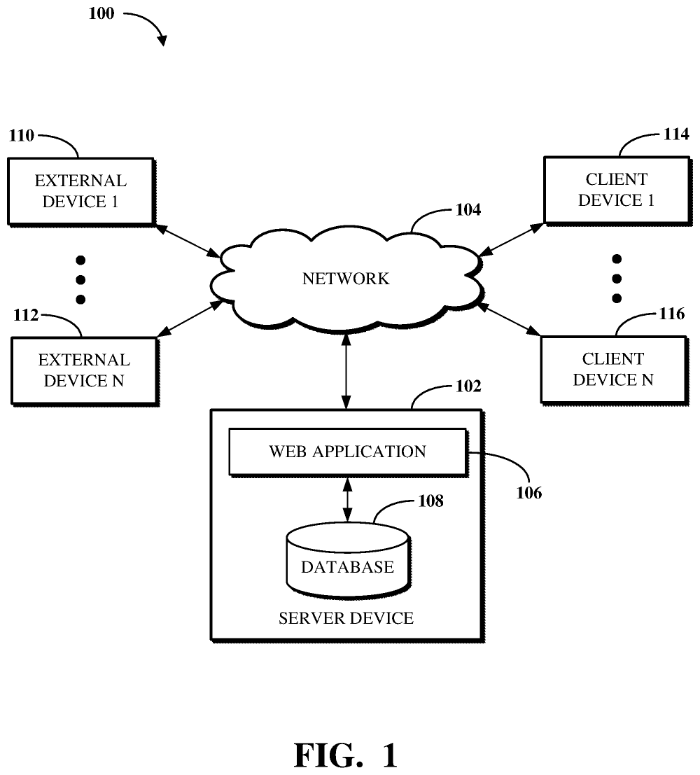

To describe some implementations in greater detail, reference is first made to examples of hardware and software structures used to implement network-implemented integrated modeling system for genetic risk estimation. FIG. 1 is a block diagram of an example of a system 100 for network-implemented integrated modeling for genetic risk estimation. The system 100 includes a server device 102 which implements integrated modeling software for genetic risk estimation over a network 104. The server device 102 is a computing aspect that runs a web application 106. The server device 102 may be or include a hardware server (e.g., a server device), a software server (e.g., a web server and/or a virtual server), or both. For example, where the server device 102 is or includes a hardware server, the server device 102 may be a server device located in a rack, such as of a data center.

The web application 106 is integrated modeling software for genetic risk estimation. In particular, the web application 106 is a network-implemented software platform which implements the integrated modeling aspects of the system 100, such as by determining models for estimating genetic risks of disease and by estimating a specific patient's genetic risk of disease using those models and patient-specific information relevant to those models. The web application 106 may be implemented in a number of different ways, for example, using a .NET platform web server running instructions or other code or script, such as written in hypertext markup language (HTML), JavaScript, and/or another language. In some implementations, the web application 106 may be software of a platform-as-a-service (PaaS) or software-as-a-service (SaaS) computing environment, such as which may implement single- or multi-tenant instances for user access.

The functionality of the web application 106 is implemented using interfaces and software tools, which use data stored in a database 108 on the server device 102. The web application 106 accesses the database 108 to perform at least some of the functionality of the web application 106. The database 108 is a database or other data store used to store, manage, or otherwise provide data used to deliver functionality of the web application 106. For example, the database 108 may be a relational database management system, an object database, an XML database, a configuration management database, a management information base, one or more flat files, other suitable non-transient storage mechanisms, or a combination thereof. The web application 106 may use structured query language (SQL) or transact-SQL (T-SQL) commands to interface with the database 108.

Functionality of the web application 106 is implemented using input information received from external devices, shown in FIG. 1 as an external device 1 110 through an external device N 112 in which N may be any integer value greater than 1. In some implementations, a single external device may provide inputs used to implement the functionality of the web application 106. In such an implementation, the external device N 112 is omitted.

The external devices may be or refer to computing devices from which some input information used by the web application 106 is received. The input information includes one or both of guideline factors used to model genetic risk or personal and/or family health histories. The guideline factors may be or refer to health data determined to be relevant for the purpose of estimating genetic risk for one or more diseases. The external devices from which the guideline factors may be received are devices operated by guideline establishing agencies, for example, the National Comprehensive Cancer Network®. The guideline factors obtained from the external devices may be referred to as first guideline factor information. As will be described below, the web application 106 may further use second guideline factor information developed for the web application 106.

The personal and/or family health histories may be or refer to data representative of a patient's personal health history and/or the patient's family members' health histories. The external devices from which the personal and/or family health histories may be received are devices operated a clinic health database or another data store of a health system facility which stores personal patient health histories, or devices used to implement an ancestral tracing service, such as which uses a database or other data store to store information for persons in a family tree and collecting health information therefor.

One or more client devices, shown in FIG. 1 as a client device 1 114 through a client device N 116 in which N may be any integer value greater than 1, may be in communication with the server device 102, such as to the use the web application 106. Each client device is a computing device configured to access the network 104 and may, for example, be a mobile device, such as a smart phone, tablet, laptop, a desktop computer, or another mobile or non-mobile computer. A client device may be used by one of multiple different types of users. For example, a client device may be used by a patient himself or herself. In another example, a client device may be used by a health care worker of a health system with a single clinic or office. In yet another example, a client device may be used by a health care worker of a health system with multiple clinics or offices.

A client device may run a software application (e.g., client-side application software) to communicate with the web application 106. For example, the software application may be a mobile application that enables access to some or all functionality and/or data of the web application 106. The software application may be a dedicated application developed to communicate with the server device 102 to interface with the web application 106. Alternatively, the software application may be a web browser application which executes hypertext transport protocol (HTTP) requests or like requests for website content and renders the website content responses to those requests.

The server device 102 communicates with the external devices and with the client devices over the network 104. The network 104 may, for example, be a local area network, a wide area network, a machine-to-machine network, a virtual private network, or another public or private network. For example, the network 104 may be the Internet. In particular, the server device 102 receives the input information used by the web application 106 from the external devices over the network 104, and the client devices access the web application 106 over the network 104. Thus, the quality of the system 100 being network-implemented as used herein refers to the use of a network for communicating information between a device which implements the genetic risk estimation functionality (e.g., the server device 102) and other devices which receive data from or transmit data to that device (e.g., the external device 1 110, the external device N 112, the client device 1 114, or the client device N 116). In some implementations, different networks may be used for communications between the server device 102 and each of the external devices and the client devices. For example, in such an implementation, the server device 102 may communicate with an external device over a first network and the server device 102 may communicate with a client device over a second network different from the first network.

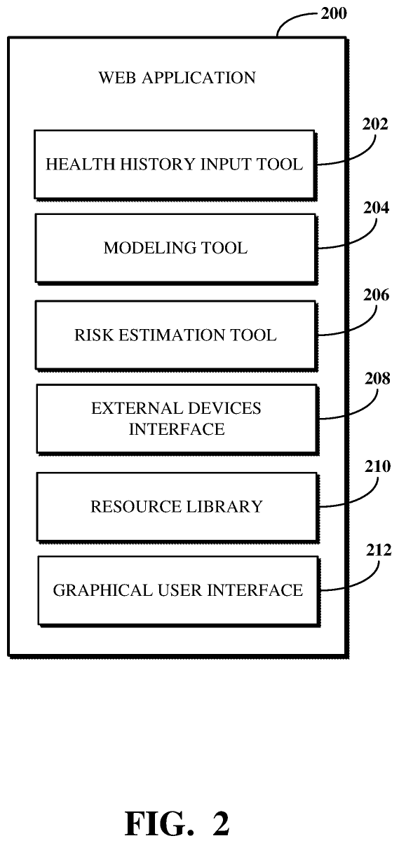

FIG. 2 is a block diagram showing examples of functionality of a web application 200 of a system for network-implemented integrated modeling for genetic risk estimation, which may, for example, be the web application 106 and the system 100 shown in FIG. 1. The web application 200 includes a health history input tool 202, a modeling tool 204, a risk estimation tool 206, an external devices interface 208, a resource library 210, and a graphical user interface (GUI) 212.

The health history input tool 202 collects health history information for a patient. The health history information is provided by the patient in response to a questionnaire transmitted to the computing device of the patient. The questionnaire includes questions designed specifically based on models determined at the web application 200, such as to capture the information which the web application 200 will use to estimate genetic risk using those models. Examples of health history information collected for a patient include, but are not limited to, dates and types of previous health screenings, smoking history, substance use history, pregnancy history (if applicable), maternal and paternal ancestry, personal diagnosis history, family member diagnosis history, ages of those family members at their time of diagnosis, and cause of death information for those family members (if applicable). The information collected using the health history input tool 202 represents personal and family health information which is stored in a database accessible by the web application, for example, the database 108 shown in FIG. 1. The patient may be asked to fill out the questionnaire more than once, such as in response to a change in guidelines used for modeling at the web application 200, in response to some amount of time elapsing since the previous time the questionnaire was filled out, both, or the like.

In some implementations, the health history input tool 202 may collect the health history information using a software wizard. The software wizard includes a series of requests for response by the patient. The software wizard may be presented to a user of the web application 200 the first time he or she accesses the web application 200. However, in some cases, the software wizard may be presented to the same user a second or subsequent time, such as to collect additional information, verify the then-current accuracy of previously provided responses, or for other purposes.

The modeling tool 204 uses certain input information received from external devices and additional information implemented at the web application 200 to model genetic risk of disease for patients. In particular, the modeling tool 204 uses guideline factors established either by an operator of the web application 200 or by a source external to the web application 200, for example, a source associated with the external device 1 110 or the external device N 112 shown in FIG. 1. For example, the modeling tool 204 may use first guideline factor information obtained from one or more external devices and/or second guideline factor information developed for the web application 200. For example, an operator of the web application 200, such as a medical researcher, health care provider, or other professional, may develop the second guideline factor information independent of the first guideline factor information. The guideline factors are used to determine manifestations, fields, and filters for selectively controlling the types of patient information used to estimate genetic risk for certain diseases and the weights, if any, applied to those types of patient information.

Manifestations are, include, or otherwise refer to categories of information which should be considered when determining a model, for example, cancer history, alcohol and other substance use history, environmental factors, genotype, phenotype, and so on. Fields are, include, or otherwise refer to the types of information within a manifestation which should be collected, for example, patient sex, patient age, location at which the patient grew up, length of time the patient was a smoker, number of family members who have been diagnosed with cancer, and so on. Filters are values of fields that are used to estimate genetic risk, for example, a number of family members who have been diagnosed with a certain type of disease, a number of environmental factors present for the patient, the patient being of at least a certain age, and so on.

The filters specifically are derived from guideline factors received at the web application 200 from an external device and/or developed for the web application 200. For example, an external source may correspond to medical researchers to have identified effective ways for estimating certain genetic disease risks by measuring values of specific patient information against criteria established by those medical researchers. The web application 200, via the modeling tool 204, determines the filters to use for a model using those guideline factors.

A model determined using the modeling tool 204 is determined for a specific disease (e.g., specific cancers, neuropsychological diseases, etc.) using manifestations, fields, and filters derived from disease-specific guideline factors. As such, the specific manifestations, fields, and filters used to determine a model for estimating a genetic risk of breast cancer may not be the same as the specific manifestations, fields, and filters used to determine a sarcoma.

In some implementations, the modeling tool 204 includes functionality for validating models determined using the modeling tool 204. For example, validating a model determined using the modeling tool 204 can include receiving, at a time after results of a risk estimation for the patient is provided by the web application 200 using the model, further information representing diagnosis and/or prognosis information for the patient. The diagnosis and/or prognosis information indicates whether the patient came to be diagnosed with the subject disease for which genetic risk was estimated and, to the extent the patient was so diagnosed, the patient's stage of disease and expected treatment outcome. The modeling tool 204 uses that further information to verify whether the results of the earlier genetic risk estimation were accurate, regardless of the specific risk indicated by those results. In the event the results are determined to be inaccurate, the modeling tool 204 can be used to reassess the model determined for the subject disease, such as to re-tune the filters, fields, or manifestations used thereby.

In some implementations, the modeling tool 204 includes functionality for updating models determined using the modeling tool 204. For example, updating a model determined using the modeling tool 204 can include determining that guideline factors used as filters in a model have changed and adjusting the filters, fields, and/or manifestations used thereby as a result. For example, updated guideline factors may be pushed to the web application 200 from an external device associated with the source of those guideline factors, such as where a configuration between the web application 200 and the external device enables such push communications when updates to the guideline factors become available. The updates are applied against the relevant model to adjust the filters, fields, and/or manifestations used thereby. In some such implementations, the updated guideline factors may be manually or automatically pulled from the external device associated with the source of those guideline factors. For example, the web application 200 may be configured to detect updates to guideline factors used by its models, such as using periodic or non-periodic methods. One example of a method for detecting an update is to use a crawler designed to search a software aspect (e.g., a website or database interface) of the source of the guideline factors for updates.

The risk estimation tool 206 uses the models determined using the modeling tool 204 to estimate the genetic risk of disease for a patient. In particular, the risk estimation tool 206 uses the personal and family health information collected using the health history input tool 202 and the models determined using the modeling tool 204 to estimate a patient's genetic risk for certain diseases associated with those models. The risk estimation tool 204 applies the values of the collected health history information against applicable criteria of a given model to estimate the genetic risk of the disease associated with that model in the patient from whom the health history information is collected. For example, the risk estimation tool 206 may classify the personal and family health information according to manifestations, fields, and filters, determine a model to use to estimate the genetic risk of the patient for the specified disease based on the classification, and apply the personal and family health information to the model to determine whether it is recommended for the patient to seek genetic counseling in regard to the specified disease.

The estimation of genetic risk for a disease using a model and health history information is specifically performed by measuring, for some or all values of the health history information, a given value against a related manifestation, field, and filter used to determine part of a model. For example, a male patient whose family member was diagnosed with breast cancer under the age of 50 may be at increased risk for aggressive melanoma, aggressive prostate cancer, or pancreatic cancer. Thus, to estimate that male patient's genetic risk of disease, the web application 200 may use a model which has manifestations corresponding to family cancer history; fields corresponding to the types of cancer, the relationship between the patient and the family members in question, and the ages of those family members at their respective times of diagnosis; and filters defined to check for diagnosis ages below 50 years.

The results of the genetic risk estimation are presented in one or more forms. For example, the risk estimation tool 206 may use the results of the estimation to generate a pedigree chart representing a multigenerational, family overview of relationships between family members of a patient who are and are not affected by a disease or condition. In another example, the risk estimation tool 206 may use the results of the estimation to generate a report including information usable by the patient or a health care provider who provides health care services to the patient to initiate a preventative treatment course of action for the patient to mitigate the risk of onset of the subject disease. The report indicates a risk level for the patient, such as indicating that the further referral of the patient for genetic counseling is at the discretion of the physician or that further genetic counseling and/or genetic testing is or is not advised for the patient, recommendations of initiation of genetic testing for certain genes, next steps for the patient to begin an applicable preventative treatment plan for the subject disease, recommendations of physicians who may be able to assist the patient in undertaking a preventative treatment plan for the subject disease, and/or other information. For example, where a patient inputs family health information indicating one or more close female relatives have been affected with a certain subtype of breast cancer, the report may advise that the patient proceed with genetic testing for certain genes known to more specifically predispose to said subtype of cancer.

The results of the genetic risk estimation performed using the risk estimation tool 206 are made available to one or more client devices which access the web application 200. For example, those results may be shared directly with the party who ordered the genetic risk estimation, such as the patient himself or herself or a health care office of a health care provider who provides health care services to the patient. In particular, where a physician or other medical professional referred the patient to use the web application 200, the results of the genetic risk estimation may be shared with that physician or other medical professional, or with the health care facility within which that physician or other medical professional works.

In some implementations, those results may, upon request by the patient and with consent from a family member of the patient, be shared with that family member, such as to alert that family member as to the estimated genetic risk assessed for the patient who used the web application 200. In some implementations, those results may, such as upon request by the patient or a current physician of the patient registered with the web application 200, be shared with another physician or medical professional, such as to whom the patient may be referred for further genetic counseling or testing.

In some implementations, the risk estimation tool 206 may include a risk calculator to further specify an estimated risk statistic calculated for the patent based on the results of his or her risk estimation. For example, the estimated risk statistic may be calculated by comparing values of the health history information collected for the patient against values of health history information for other patients, whether or not registered with the web application 200, who have also been previously determined to be at risk for a subject disease and have later been diagnosed with the subject disease. For example, the estimated risk statistic may be calculated based on similarities in values of the health history information of the patient and the health history information of those other patients.

The external devices interface 208 includes a set of instructions and/or configurations usable to interface with external devices from which input information used by the web application 200 is received, for example, the external device 1 110 or the external device N 112 shown in FIG. 1. For example, the external devices interface 208 may include instructions and/or configurations for using the health level seven (HL7) standard for clinical and administrative health data communications, such as of personal and/or family health information, to or from a server device implementing the web application 200, for example, the server device 102 shown in FIG. 1. In another example, the external devices interface 208 may include instructions and/or configurations for using an application programming interface (API) of a web service from which guideline factors or family health information may be received.

The resource library 210 includes a collection of resources related to diseases for which a patient's genetic risk may be estimated. The resources of the resource library 210 may be generated at the web application 200 or received from external sources, such as the external device 1 110 or the external device N 112. The resources of the resource library 210 include educational materials, research articles, health care provider contact information, and other documents and information. The resource library 210 may generally be accessible in full to a user of the web application 200. However, results determined by the risk estimation tool 206 for a patient may be used to highlight, recommend, or otherwise direct user attention to certain specific resources of the resource library 210 which are deemed relevant to those results.

For example, a resource of the resource library 210 may be or include general information about informed consent for patients, family health history importance in genetic counseling, personalized medicine, and/or other topics. In another example, a resource of the resource library 210 may be or include information specific to a disease for which a genetic risk has been indicated to a patient, such as information about a type of cancer and its early onset signs, mitigating steps the patient can take to decrease his or her genetic risk, health concerns associated with a diagnosis of the subject disease, and/or other aspects.

The GUI 212 is a GUI which may be rendered or displayed, such as to render or display pages of the web application 200 for use at client devices, for example, the client device 1 114 or the client device N 116 shown in FIG. 1. The GUI 212 can comprise part of a software GUI constituting data that reflect information ultimately destined for display on a hardware device, for example, on one of the client device 1 114 or the client device N 116. For example, the data can contain rendering instructions for bounded graphical display regions, such as windows, or pixel information representative of controls, such as buttons and drop-down menus. The rendering instructions can, for example, be in the form of HTML, standard generalized markup language (SGML), JavaScript, Jelly, AngularJS, or other text or binary instructions for generating the GUI 214 or another GUI on a display that can be used to generate pixel information. A structured data output of one device can be provided to an input of the hardware display so that the elements provided on the hardware display screen represent the underlying structure of the output data.

In some implementations, the web application 200 may include additional software tools and other features for implementing further functionality beyond what is described above with respect to FIG. 2. For example, in some implementations, the web application 200 may include a machine learning tool. For example, the machine learning tool may be trained to enhance the functionality of the web application 200 using a training data set including information associated with the models determined using the modeling tool 204 and information associated with the results of the risk estimation tool 204. The machine learning tool may then perform one or more types of inference against information processed by the web application 200 such as to improve or automate functionality of the web application 200. In some such implementations, the machine learning tool may collect further information received from patients at a time after initial results of genetic risk estimation are presented, such as one year later, to understand whether any further signs of the subject disease have presented in the patient, whether any changes in health history information of the patient are relevant for re-estimating the genetic risk for the disease, or to understand whether new health information not previously collected may be relevant to the genetic risk estimation process.

In some such implementations, the machine learning tool may use those pieces of further information to determine statistical correlations between the criteria which is relevant for estimating genetic risk and the high value of that criteria to that estimation. Similarly, in some such implementations, the machine learning tool may use those pieces of further information to determine other statistical correlations between the criteria which is least relevant for estimating genetic risk and the low value of that criteria to that estimation. In either way, the questionnaire designed to collect patient health history information may be redesigned to more effectively collect the information which will be of the greatest use for estimating genetic risk of disease.

In another example, in some implementations, the web application 200 may include administrative portals for a health system user of the web application 200 which refers patients to the web application 200 for genetic risk estimation and/or for an entity which operates the web application 200. For example, the administrative portals may enable the selective configuration of certain features of the web application 200, enable to customization of the aesthetics of the web application 200, and/or provide logistical and other measured data regarding usage of the web application 200.

In yet another example, in some implementations, the web application 200 may include a physician portal for health care professionals to access to review results of genetic risk estimations performed at the web application 200. The physician portal may further include functionality for a health care professional to generate a referral for a patient to use the web application 200, such as which may cause an invitation to be communicated to the patient at a stated contact entity. The patient may responsively accept the invitation by accessing the web application 200 and beginning the workflow for estimating the patient's genetic risk. The physician portal may further include functionality for the health care professional to order tests from local medical facilities based on the results of a patient's genetic risk estimation.

In some implementations, the physician portal may include functionality for allowing a health care professional to refer a patient's records with the web application 200 to another health care professional. For example, if the results of the genetic risk estimation for a patient indicate that the patient is at risk for pancreatic cancer, the patient's primary care physician may use the physician portal of the web application 200 to generate a referral for the patient to be seen by a gastroenterologist.

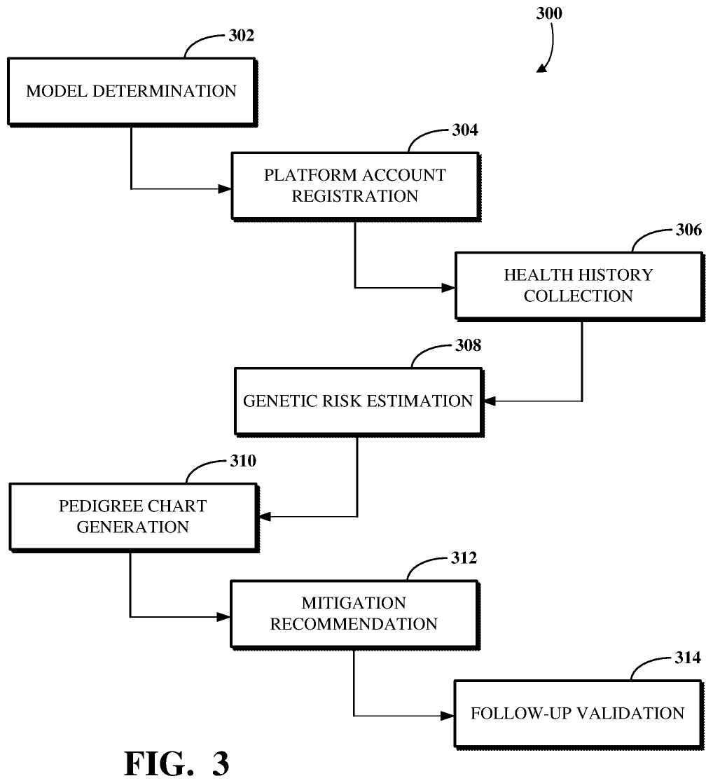

FIG. 3 is a block diagram of an example workflow 300 of a network-implemented integrated modeling system for genetic risk estimation, for example, the system 100 shown in FIG. 1. The workflow 300 begins with a model determination stage 302 at which models usable for estimating genetic risk of disease are determined. The models may be determined using manifestations, fields, and filters derived from first guideline factor information received from sources external to a web application which implements the workflow 300, for example, the web application 200 shown in FIG. 2, and/or second guideline factor information developed for the web application.

At a time after the models are determined, the workflow 300 next goes to a platform account registration stage 304 at which a patient registers an account with the platform of the web application which implements the workflow 300. The account registration may be at the patient's own election. Alternatively, the account registration may be responsive to an invitation to register the account, such as based on an invitation to do so from a health care provider or other health care representative who cares for the patient.

After the patient registers his or her account with the platform of the web application, the workflow 300 proceeds to a health history collection stage 306 which collects personal and/or family health information. The specific questions included in the questionnaire or the software wizard, as applicable based on the implementation, are defined using the guideline factors earlier used to determine the models, and thus the specific questions are requests for information corresponding to the manifestations, fields, and filters used as part of a model for a given disease.

The workflow 300 then proceeds to a genetic risk estimation stage 308 at which the health history information collected at the health history collection stage 306 is processed against one or more models determined at the model determination stage 302 to estimate the genetic risk of the patient for the diseases associated with those one or more models. Processing the health history information of the patient using a model includes assessing certain aspects of the health history information according to the definitions of the model, for example, the filters used to determine the model. The web application may by default select to process the health history information of the patient against each of the models in order to estimate the genetic risk of all diseases for which the web application is configured. Alternatively, the patient may indicate the disease or diseases for which the patient would like to have his or her genetic risk estimated.

The results of the genetic risk estimation stage 308 are used at a pedigree chart generation stage 310 of the workflow 300 to generate a pedigree chart for the patient. The pedigree chart shows relationships between a patient for whom genetic risk is estimated and the patient's family members. In particular, the pedigree chart is a visual representation of relationships between the patient and one or more of his or her family members, who may or may not share the same disease risk or who may, based on family health information collected at the web application, have a different genetic disease risk.

The workflow 300 proceeds to a mitigation recommendation stage 312 at which other reporting accompanying the pedigree chart is generated, for example, to report the diseases for which the patient may be estimated have genetic risk, a recommendation for the patient to seek further genetic counseling based on the genetic risk estimation, a recommendation for the patient to seek genetic testing for mutations relevant to diseases for which the patient may be estimated to have genetic risk, steps to initiate a preventative treatment plan for mitigating the estimated genetic risk with a health care professional, and/or the like. The other reporting may be presented in a same output which also includes the pedigree chart. Alternatively, the pedigree chart and the other reporting may be separately provided to the patient or a health care provider who provides health care services for the patient.

As a final step of the workflow 300, a follow-up validation stage 314 collects further information at a time after the genetic risk of the patient is first estimated, for example, six months or one year later. The further information is intended to determine whether the web application should re-estimate the genetic risk of one or more diseases for the patient, such as based on changes to the patient's health and/or changes to the guideline factors underlying the models used to estimate the genetic risks. In the event further follow-up is warranted, an updated questionnaire or an updated software wizard, as applicable per the particular implementation, of the web application is used to collect further health information from the patient. That new health information is used to re-estimate the genetic risk of the patient using one or more applicable models. In some cases, the follow-up validation stage 314 may use the new health information to re-tune the models, such as based on statistical information indicating that certain health information collected by patients tends to be immaterial for the purpose of estimating the genetic risk of a disease associated with a given model.

Aspects of the workflow 300 may be repeated in some cases, such as based on updates to the first guideline factor information, the second guideline factor information, and/or the personal and family health information. For example, based on a change to one of the first guideline factor information or the second guideline factor information, one or more manifestations, fields, or filters of a model may be updated to produce an updated model. An updated genetic risk of the patient for a specified disease may then be estimated using the updated model and using the personal and family health information. An updated pedigree chart and/or updated reporting data may then be generated based on results of the updated genetic risk estimation and distributed to one or more client devices. In another example, a genetic risk estimation may be repeated using an initial model responsive to further personal and family health information being received, such as from a patient who previously provided the initial personal and family health information. For example, the further personal and family health information may represent changes to the patient's personal and/or family health history which may be relevant for the purpose of estimating a genetic risk of disease for the patient.

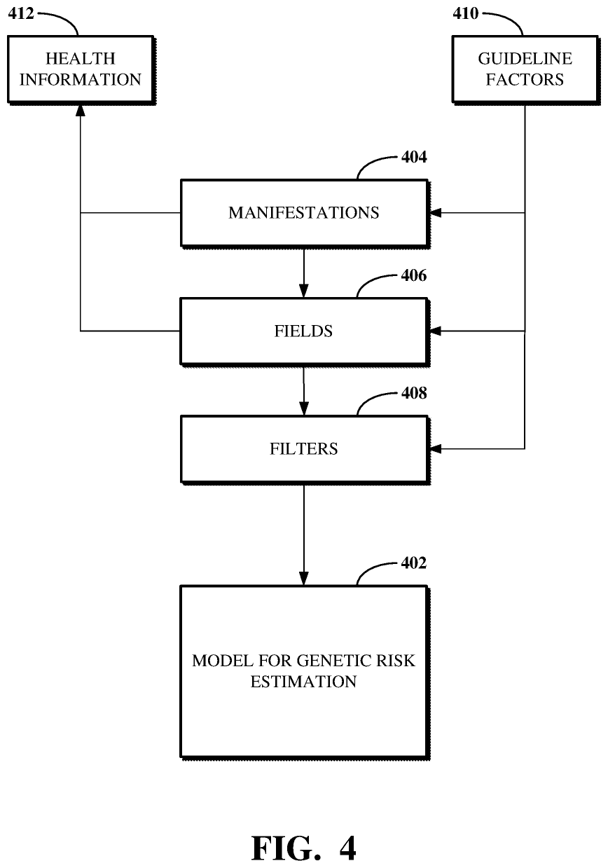

FIG. 4 is a block diagram of an example of inputs and criteria used for modeling genetic risk. As previously stated, a model for genetic risk estimation 402, which may, for example, be a model determined using the modeling tool 204 of the web application 200 shown in FIG. 2, is determined using manifestations 404, fields 406, and filters 408. The manifestations 404, fields 406, and filters 408 derive from guideline factors 410 received at the web application from external sources and/or developed for the web application. In particular, the guideline factors 410 indicate the types of health history information 412 which should be collected from a patient who uses the web application. That is, the guideline factors 410 specify the filters 408 by establishing certain values or value ranges of types of information which are usable to estimate genetic risk for disease. The filters 408 specified by the guideline factors 410 thus indicate the manifestations into which portions of the health history information 412 is classified fields 406 for the manifestations 404.

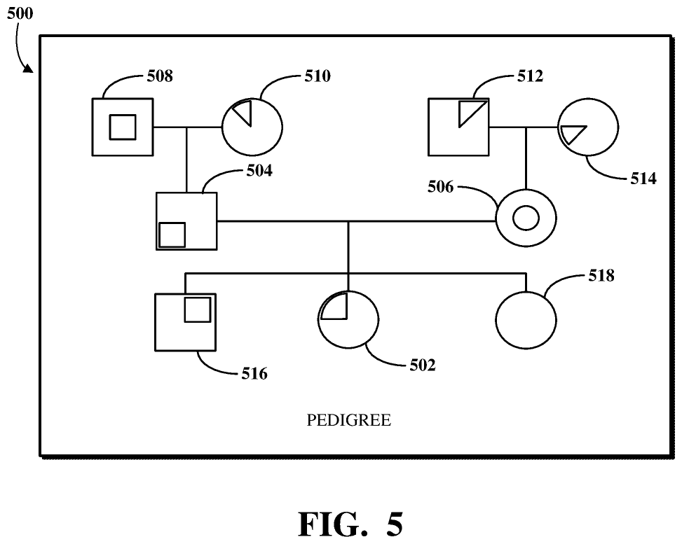

FIG. 5 is an illustration of a pedigree chart 500 generated for a patient. For example, the pedigree chart 500 may represent one form of output from a risk estimation tool of a web application in a network-implemented integrated modeling system for genetic risk estimation, such as the risk estimation tool 206 of the web application 200 shown in FIG. 2.

A pedigree chart generated within a network-implemented integrated modeling system for genetic risk estimation according to this disclosure shows family relationships between a patient for whom genetic risk is estimated and the patient's family members. In the example shown in FIG. 5, the pedigree chart 500 shows a proband representing a patient 502 having a father 504, a mother 506, a paternal grandfather 508, a paternal grandmother 510, a maternal grandfather 512, a maternal grandmother 514, a brother 516, and a sister 518. Other relatives with various degrees of relation may be included in a pedigree chart.

Although the pedigree chart 500 shows three generations of family members, a pedigree chart generated according to the implementations of this disclosure may include more generations or fewer generations. For example, a pedigree chart generated according to the implementations of this disclosure may include seven generations of which three are above the proband representing the patient and three below the proband representing the patient. In particular, a pedigree chart generated according to the implementations of this disclosure will, if possible, show one or more generations below a proband representing the patient (e.g., the patient 502) so as to indicate potentially already known disease risks or diagnoses of specific diseases, whether they be hereditary or not, for family members of those one or more generations below the proband.

The shapes of the persons represented in the pedigree chart 500 indicate the sex of those persons. In the example shown, a circle represents the subject person as being female and a square represents the subject person as being male. Sub-shapes included within a circle or square represent identified potentially already known disease risks or diagnoses of specific diseases, whether they be hereditary or not. The type of sub-shape and/or the location of a sub-shape within a circle or square may be defined to correspond to a specific disease. In some implementations, a legend may be provided along with the pedigree chart 500 as output to indicate the specific indications associated with the sub-shapes shown.

For example, the quarter pie sub-shape in the upper left of the patient 502 indicates a risk or diagnosis of breast cancer, the square sub-shape in the upper right of the brother 516 indicates a risk or diagnosis of colorectal cancer, the square sub-shape in the lower left of the father 504 indicates a risk or diagnosis of prostate cancer, the circle sub-shape in the middle of the mother 506 indicates a risk or diagnosis of pancreatic cancer, the square sub-shape in the middle of the paternal grandfather 508 indicates a risk or diagnosis of sarcoma, the eighth pie sub-shape in the upper left of the paternal grandmother 510 indicates a risk or diagnosis of uterine cancer, the triangle in the upper right of the maternal grandfather 512 indicates a risk or diagnosis of a gastric disorder, and the quarter pie in the lower left of the maternal grandmother 514 indicates a risk or diagnosis of endometrial cancer.

The pedigree chart 500 thus represents specific family risk of disease types, which may benefit the patient 502 in better understanding the specific nature of his or her genetic risk of certain diseases, but which may also indicate family members who may themselves benefit from genetic counseling. The pedigree chart 500 may be included in output from the web application to a health care provider of one or more of the family members shown in the pedigree 500, such as to indicate the estimated disease risk thereof. In some implementations, the web application may, with the consent of the family members listed on the pedigree chart 500, be configured to automatically share results of the pedigree chart 500 with those family members and/or with identified health care professionals who provide health care services for those family members.

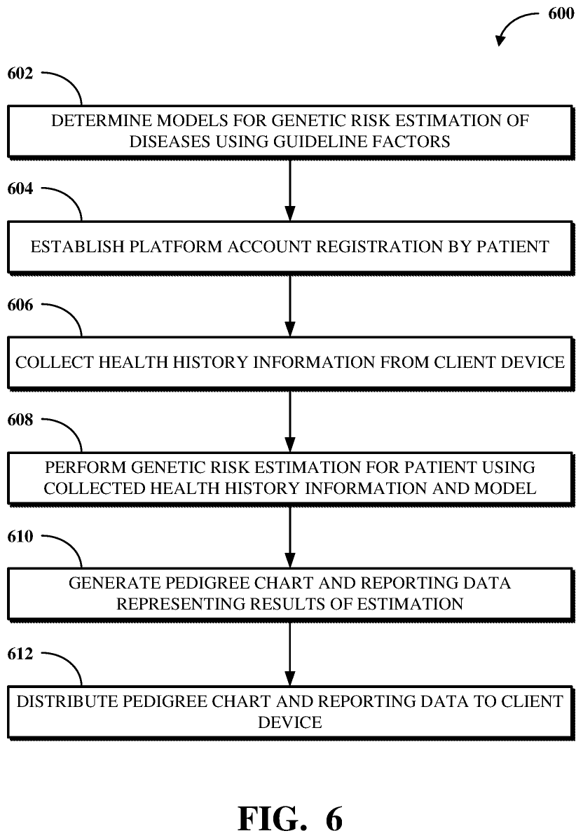

To further describe some implementations in greater detail, reference is next made to examples of techniques which may be performed by or using network-implemented integrated modeling system for genetic risk estimation, such as described with respect to FIGS. 1-5. FIG. 6 is a flowchart of an example of a technique 600 for genetic risk estimation. The technique 600 can be executed using computing devices, such as the systems, hardware, and software described with respect to FIGS. 1-5. The technique 600 can be performed, for example, by executing a machine-readable program or other computer-executable instructions, such as routines, instructions, programs, or other code. The steps, or operations, of the technique 600 or another technique, method, process, or algorithm described in connection with the implementations disclosed herein can be implemented directly in hardware, firmware, software executed by hardware, circuitry, or a combination thereof.

For simplicity of explanation, the technique 600 is depicted and described herein as a series of steps or operations. However, the steps or operations in accordance with this disclosure can occur in various orders and/or concurrently. Additionally, other steps or operations not presented and described herein may be used. Furthermore, not all illustrated steps or operations may be required to implement a technique in accordance with the disclosed subject matter.

At 602, models for genetic risk estimation of diseases are determined using first guideline factor information and/or second guideline factor information. At 604, platform account registration is established by a patient. At 606, health history information of the patient is collected at a web application implementing the network-implemented integrated modeling. At 608, genetic risk estimation is performed for the patient using the collected health history information and a model determined using the guideline factors. At 610, a pedigree chart and other reporting data representing the results of the genetic risk estimation are generated. At 612, the pedigree chart and other generated reporting data are distributed to one or more client devices in communication with the web application over a network.

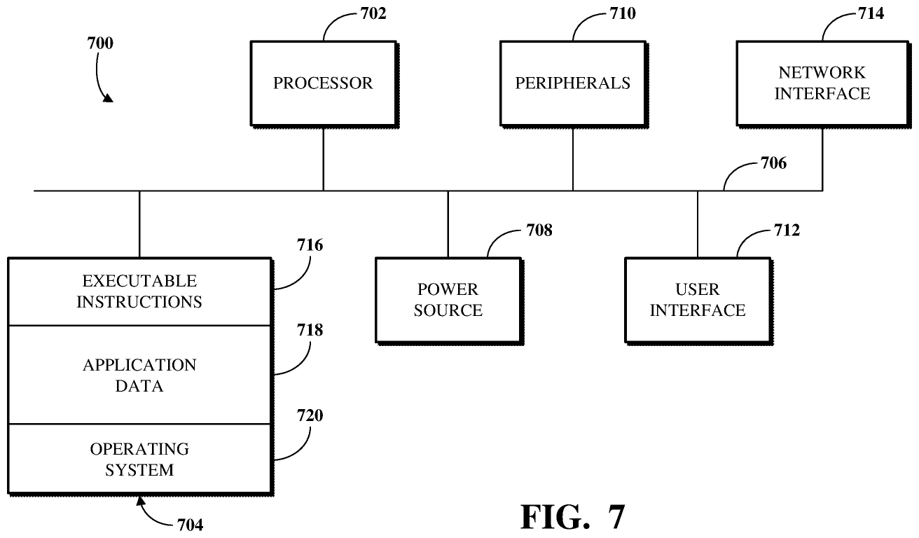

FIG. 7 is a block diagram of an example internal configuration of a computing device 700 of a system for network-implemented integrated modeling for genetic risk estimation, for example, the system 100 shown in FIG. 1. The computing device 700 may be used to implement a server on which a web application is run (e.g., the server device 102 and the web application 106 shown in FIG. 1). Alternatively, the computing device 700 may be used to implement a client that accesses the web application (e.g., the client device 1 114 or the client device N 116 shown in FIG. 1). As a further alternative, the computing device 700 may be used as or to implement another client, server, or other device according to the implementations disclosed herein.

The computing device 700 includes components or units, such as a processor 702, a memory 704, a bus 706, a power source 708, peripherals 710, a user interface 712, and a network interface 714. One or more of the memory 704, the power source 708, the peripherals 710, the user interface 712, or the network interface 714 can communicate with the processor 702 via the bus 706.

The processor 702 is a central processing unit, such as a microprocessor, and can include single or multiple processors having single or multiple processing cores. Alternatively, the processor 702 can include another type of device, or multiple devices, now existing or hereafter developed, configured for manipulating or processing information. For example, the processor 702 can include multiple processors interconnected in any manner, including hardwired or networked, including wirelessly networked. For example, the operations of the processor 702 can be distributed across multiple devices or units that can be coupled directly or across a local area or other suitable type of network. The processor 702 can include a cache, or cache memory, for local storage of operating data or instructions.

The memory 704 includes one or more memory components, which may each be volatile memory or non-volatile memory. For example, the volatile memory of the memory 704 can be random access memory (RAM) (e.g., a DRAM module, such as DDR SDRAM) or another form of volatile memory. In another example, the non-volatile memory of the memory 704 can be a disk drive, a solid state drive, flash memory, phase-change memory, or another form of non-volatile memory configured for persistent electronic information storage. The memory 704 may also include other types of devices, now existing or hereafter developed, configured for storing data or instructions for processing by the processor 702.

The memory 704 can include data for immediate access by the processor 702. For example, the memory 704 can include executable instructions 716, application data 718, and an operating system 720. The executable instructions 716 can include one or more application programs, which can be loaded or copied, in whole or in part, from non-volatile memory to volatile memory to be executed by the processor 702. For example, the executable instructions 716 can include instructions for performing some or all of the techniques of this disclosure. The application data 718 can include user data, database data (e.g., database catalogs or dictionaries), or the like. The operating system 720 can be, for example, Microsoft Windows®, Mac OS X®, or Linux®, an operating system for a small device, such as a smartphone or tablet device; or an operating system for a large device, such as a mainframe computer.

The power source 708 includes a source for providing power to the computing device 700. For example, the power source 708 can be an interface to an external power distribution system. In another example, the power source 708 can be a battery, such as where the computing device 700 is a mobile device or is otherwise configured to operate independently of an external power distribution system.

The peripherals 710 includes one or more sensors, detectors, or other devices configured for monitoring the computing device 700 or the environment around the computing device 700. For example, the peripherals 710 can include a geolocation component, such as a global positioning system location unit. In another example, the peripherals can include a temperature sensor for measuring temperatures of components of the computing device 700, such as the processor 702.

The user interface 712 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 714 provides a connection or link to a network (e.g., the network 116 shown in FIG. 1). The network interface 714 can be a wired network interface or a wireless network interface. The computing device 700 can communicate with other devices via the network interface 714 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

In some implementations, the computing device 700 can omit the peripherals 710. In some implementations, the memory 704 can be distributed across multiple devices. For example, the memory 704 can include network-based memory or memory in multiple clients or servers performing the operations of those multiple devices. In some implementations, the application data 718 can include functional programs, such as a web browser, a web server, a database server, another program, or a combination thereof.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc.

Likewise, the terms “system” or “mechanism” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

1. A network-implemented integrated modeling system for genetic risk estimation, comprising:
a server device implementing a web application accessible over a network by a client device and a database storing information including a first set of guideline factor information obtained from one or more external sources and a second set of guideline factor information developed for the web application,
wherein the web application is configured to:
derive, using one or more processors of the server device, manifestations, fields, and filters for selectively controlling types of patient information used to estimate genetic risk for one or more specified diseases based on the first set of guideline factor information and the second set of guideline factor information;
determine, using the one or more processors of the server device, models for estimating the genetic risk of the one or more specified diseases based on the manifestations, fields, and filters;
transmit, using the one or more processors of the server device, a request for personal and family health information associated with a patient to the client device over the network;
receive, using the one or more processors of the server device, the personal and family health information from the client device over the network;
estimate, using the one or more processors of the server device, a genetic risk of the patient for each of the one or more specified diseases at a first time by evaluating the personal and family health information against the models;
generate, using the one or more processors of the server device, a pedigree chart and reporting data based on results of the genetic risk estimation at the first time, the pedigree chart visually representing relationships between the patient and family members of the patient, the reporting data indicating actions for the patient to take based on the results of the genetic risk estimation;
distribute, using the one or more processors of the server device, the pedigree chart and the reporting data to the client device over the network;
automatically detect, using the one or more processors of the server device, a change to one or both of the first set of guideline factor information or the second set of guideline factor information at a second time after the first time;
derive, using the one or more processors of the server device, updated manifestations, fields, and filters based on the detected change to the one or both of the first set of guideline factor information or the second set of guideline factor information;
update, using the one or more processors of the server device, one or more of the models based on the updated manifestations, fields, and filters;
estimate, using the one or more processors of the server device, the genetic risk of the patient for each of the one or more specified diseases at the second time by evaluating the personal and family health information against the updated models;
generate, using the one or more processors of the server device, an updated pedigree chart and updated reporting data based on results of the genetic risk estimation at the second time; and
automatically distribute, using the one or more processors of the server device, the updated pedigree chart and the updated reporting data to the client device over the network.

2. The network-implemented integrated modeling system of claim 1, wherein
the manifestations represent categories of health information,
the fields represent types of specific health information in the categories, and
the filters represent definitions for values of the specific health information.

3. The network-implemented integrated modeling system of claim 1, wherein, to estimate the genetic risk of the patient for the one or more specified diseases at the first time, the web application is configured to:
classify the personal and family health information according to the manifestations, the fields, and the filters;
determine to use the model to estimate the genetic risk of the patient for the specified disease based on the classification; and
apply the personal and family health information to the model to determine whether it is recommended for the patient to seek genetic counseling in regard to the specified disease.

4. (canceled)

5. The network-implemented integrated modeling system of claim 1, wherein the web application is further configured to:
transmit, over the network, a request for further personal and family health information based on the detected change to the first guideline factor information; and
receive, over the network, the further personal and family health information from the client device,
wherein the updated genetic risk estimation is performed using the further personal and family health information.

6. (canceled)

7. The network-implemented integrated modeling system of claim 1, wherein the client device is a first client device associated with the patient, wherein the web application is further configured to:
distribute, over the network, the pedigree chart and the reporting data to a second client device associated with a health care facility of a health care provider who provides health care service to the patient.

8. The network-implemented integrated modeling system of claim 7, wherein the web application is further configured to:
receive, over the network, additional patient information from the second client device, the additional patient information including electronic patient record information associated with the patient, and
wherein the genetic risk of the patient for the one or more specified diseases is estimated using the additional patient information.

9. The network-implemented integrated modeling system of claim 7, wherein the web application is further configured to:
receive, over the network, a request for genetic risk estimation for the patient from the second client device; and
transmit, over the network, an invitation for the genetic risk estimation to the first client device responsive to the request,
wherein the request for the personal and family health information is transmitted to the first client device based on an acceptance of the invitation.

10. The network-implemented integrated modeling system of claim 7, wherein the web application is further configured to:
receive, over the network, a referral request for the patient to be seen by another health care provider from the second client device; and
transmit, over the network, information indicative of the referral request and the other health care provider to the first client device.

11. The network-implemented integrated modeling system of claim 1, wherein the database further stores resource information available to the client device via the web application, wherein the web application is further configured to:
transmit, over the network, a recommendation for at least some of the resource information to the client device based on the estimation of the genetic risk of the patient for the one or more specified diseases, the at least some of the resource information corresponding to a specified disease of the one or more specified diseases.

12. The network-implemented integrated modeling system of claim 1, wherein the pedigree chart indicates known disease risks or diagnoses for family members of one or more generations below a proband representing the patient.

13. A network-implemented integrated modeling system for genetic risk estimation, comprising:
a server device implementing a web application accessible over a network by a client device and a database storing information including guideline factor information,
wherein the web application is configured to:
derive, using one or more processors of the server device, manifestations, fields, and filters for selectively controlling types of patient information used to estimate genetic risk for one or more specified diseases based on the guideline factor information;
determine, using the one or more processors of the server device, models for estimating the genetic risk of the one or more specified diseases based on the manifestations, fields, and filters derived from the guideline factor information;
collect, using the one or more processors of the server device, personal and family health information associated with a patient from the client device;
estimate, using the one or more processors of the server device, a genetic risk of the patient for each of the one or more specified diseases at a first time by evaluating the personal and family health information against the models;
generate, using the one or more processors of the server device, a pedigree chart and reporting data based on results of the genetic risk estimation at the first time;
distribute, using the one or more processors of the server device, the pedigree chart and the reporting data to the client device;
automatically detect, using the one or more processors of the server device, a change to the guideline factor information at a second time after the first time;
derive, using the one or more processors of the server device, updated manifestations, fields, and filters based on the detected change to the guideline factor information;
update, using the one or more processors of the server device, one or more of the models based on the updated manifestations, fields, and filters;
estimate, using the one or more processors of the server device, the genetic risk of the patient for each of the one or more specified diseases at the second time by evaluating the personal and family health information against the updated models;
generate, using the one or more processors of the server device, an updated pedigree chart and updated reporting data based on results of the genetic risk estimation at the second time; and
automatically distribute, using the one or more processors of the server device, the updated pedigree chart and the updated reporting data to the client device over the network.

14. The network-implemented integrated modeling system of claim 13, wherein the manifestations represent categories of health information, the fields represent types of specific health information in the categories, and the filters represent definitions for values of the specific health information.

15. The network-implemented integrated modeling system of claim 13, wherein the guideline factor information includes first guideline factor information obtained from one or more external devices and second guideline factor information developed for the web application.

16. The network-implemented integrated modeling system of claim 13, wherein the pedigree chart and the reporting data are further distributed to a second client device associated with a health care facility of a health care provider who provides health care service to the patient.

17. A network-implemented integrated modeling system for genetic risk estimation, comprising:
a server device implementing a web application and a database storing information including first guideline factor information obtained from one or more external sources and second guideline factor information developed for the web application, wherein the web application is accessible over a network by each of a first client device associated with a patient and a second client device associated with a health care provider who provides health care service to the patient,
wherein the web application is configured to:
derive, using one or more processors of the server device, manifestations, fields, and filters for selectively controlling types of patient information used to estimate genetic risk for one or more specified diseases based on the first set of guideline factor information and the second set of guideline factor information;
determine, using the one or more processors of the server device, models for estimating the genetic risk of the one or more specified diseases based on the manifestations, fields, and filters;
estimate, using the one or more processors of the server device, a genetic risk of the patient for each of the specified diseases by evaluating personal and family health information associated with the patient against the models determined at the web application using the first guideline factor information and the second guideline factor information;
generate, using the one or more processors of the server device, results of the genetic risk estimation indicating whether further genetic counseling or testing for the specified disease is recommended; and
distribute, using the one or more processors of the server device, the results of the genetic risk estimation to the first client device and to the second client device over the network;
automatically detect, using the one or more processors of the server device, a change to one or both of the first set of guideline factor information or the second set of guideline factor information at a second time after the first time;
derive, using the one or more processors of the server device, updated manifestations, fields, and filters based on the detected change to the one or both of the first set of guideline factor information or the second set of guideline factor information;
update, using the one or more processors of the server device, one or more of the models based on the updated manifestations, fields, and filters;
estimate, using the one or more processors of the server device, the genetic risk of the patient for each of the one or more specified diseases at the second time by evaluating the personal and family health information against the updated models;
generate, using the one or more processors of the server device, results of the genetic risk estimation at the second time; and
automatically distribute, using the one or more processors of the server device, the results of the genetic risk estimation at the second time to the first client device and the second client device over the network.

18. The network-implemented integrated modeling system of claim 17, wherein
the manifestations represent categories of health information,
the fields represent types of specific health information in the categories, and
the filters represent definitions for values of the specific health information.

19. The network-implemented integrated modeling system of claim 17, wherein the results of the genetic risk estimation at the first time include a pedigree chart visually representing relationships between the patient and family members of the patient, and wherein the pedigree chart indicates known disease risks or diagnoses for family members of one or more generations below a proband representing the patient.

20. (canceled)

21. The network-implemented integrated modeling system of claim 17, wherein the web application is further configured to receive, over the network, additional patient information from the second client device, the additional patient information including electronic patient record information associated with the patient, and
wherein the genetic risk of the patient for the one or more specified diseases is estimated using the additional patient information.

22. The network-implemented integrated modeling system of claim 17, wherein the web application is further configured to:
receive, over the network, a referral request for the patient to be seen by another health care provider from the second client device; and
transmit, over the network, information indicative of the referral request and the other health care provider to the first client device.

23. The network-implemented integrated modeling system of claim 17, wherein the database further stores resource information available to the client device via the web application, wherein the web application is further configured to:
transmit, over the network, a recommendation for at least some of the resource information to the client device based on the estimation of the genetic risk of the patient for the one or more specified diseases, the at least some of the resource information corresponding to a specified disease of the one or more specified diseases.
